(19) United States
(12) Patent Application Publication
(10) Pub. No.: US 2008/0255606 A1
(43) Pub. Date: Oct. 16, 2008

(54) FILTERING DEVICE FOR USE WITHIN A BODY LUMEN

(75) Inventors: Ashish Mitra, Santa Rosa, CA (US); Meena Ramachandran, San Francisco, CA (US); Karan Punga, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/735,550

(22) Filed: Apr. 16, 2007

(51) Int. Cl. A61F 2/01

(52) U.S. Cl. 606/200

(57) ABSTRACT

A filtering apparatus for collecting emboli or thromboli in a body lumen. The apparatus may be a temporary filtering device mounted on a core wire for use during interventional procedures or a semi-permanent filtering device that is left within a body lumen for a therapeutically appropriate length of time. The filtering device includes a tubular member having an outer surface for apposition with the body lumen and a filter member positioned within an interior of the tubular member. The fitter member includes an apex at a proximal end that is positioned along a longitudinal axis of the filtering device, a distal end attached to the tubular member, and an outer surface facing an inner surface of the tubular member.

### FIELD OF THE INVENTION

The invention relates generally to intraluminal filtering devices for capturing particulate in the vessels of a patient. More particularly, the invention relates to a filtering device for capturing emboli and/or thromboli in a blood vessel.

### BACKGROUND OF THE INVENTION

Catheters have long been used for the treatment of diseases of the cardiovascular system, such as treatment or removal of stenosis. For example, in a percutaneous transluminal coronary angioplasty (PTCA) procedure, a catheter is used to transport a balloon into a patient's cardiovascular system, position the balloon at a desired treatment location, inflate the balloon, and remove the balloon from the patient. Another example of a common catheter-based treatment is the placement of an intravascular stent in the body on a permanent or semi-permanent basis to support weakened or diseased vascular walls, or to avoid closure, re-closure or rupture thereof.

These non-surgical interventional procedures often avoid the necessity of major surgical operations. However, one common problem associated with these procedures is the potential release of debris into the bloodstream that can occlude or embolize distal vasculature and cause significant health problems to the patient. For example, during deployment of a stent, it is possible for the metal struts of the stent to cut into the stenosis and shear off pieces of atherosclerotic plaque which become embolic debris that can travel downstream and lodge somewhere in the patient's vascular system. Further, pieces of plaque or clot material can sometimes dislodge from the stenosis during a balloon angioplasty procedure and become entrained in the bloodstream.

Medical devices have been developed to attempt to deal with the problem created when debris or fragments are dislodged in the circulatory system during vessel treatment. One technique includes the temporary placement of an intravascular filter or trap downstream from the treatment site to capture debris before it can reach and embolize smaller blood vessels downstream. The placement of a filter in the patient's vasculature during treatment of a vascular lesion can collect embolic debris in the bloodstream. At the end of the vessel treatment, the filter can be removed along with the captured debris. Such filters typically comprise a filtration membrane, mesh or “basket” having a plurality of pores, each pore being sized to prevent passage of particulate larger than a certain size, e.g., 100-200 microns. One micron equals one millionth or 10⁻⁶ of a meter. It is understood that a particle collected in a filter lends to lodge across a pore, substantially blocking blood flow through that pore. Therefore, the total blood flow through all the pores of a filter wilt diminish as more particulate is collected in the filter and more pores become clogged.

It is known to attach an expandable filter to a distal end of a guidewire or guidewire-like member that allows the filtering device to be placed in the patient's vasculature. The guidewire allows the physician to steer the filter to a location downstream from the area of treatment. Once the guidewire is in proper position in the vasculature, the embolic filter can be deployed to capture embolic debris. Some embolic filtering devices utilize a restraining sheath to maintain the expandable filter in its collapsed configuration while it is located. When the proximal end of the restraining sheath is retracted by the physician, the expandable filter will transform into its fully expanded configuration in apposition with the vessel wall. The restraining sheath can then be removed from the guidewire allowing the guidewire to be used by the physician to deliver interventional devices, such as a balloon angioplasty catheter or a stent delivery catheter, into the area of treatment. After the interventional procedure is completed, a recovery sheath can be delivered over the guidewire using over-the-wire techniques to collapse the expanded filter (with the trapped embolic debris) for removal from the patient's vasculature. Both the delivery sheath and recovery sheath should be relatively flexible to track over the guidewire and to avoid straightening the body vessel once in place.

Another distal protection device known in the art includes a filter mounted on a distal portion of a hollow guidewire or tube. A moveable core wire is used to open and close the filter. The filter is coupled at a proximal end to the tube and at a distal end to the core wire. Pulling on the core wire while pushing on the tube draws the ends of the filter toward each other, causing the filter framework between the ends to expand outward into contact with the vessel wall. Filter mesh material is mounted to the filter framework. To collapse the filter, the procedure is reversed, i.e., pulling the tube proximally while pushing the core wire distally to force the filter ends apart. A sheath catheter may be used as a retrieval catheter at the end of the interventional procedure to reduce the profile of the “push-pull” filter, as due to the embolic particles collected, the filter may still be in a somewhat expanded state. The retrieval catheter may be used to further collapse the filter and/or smooth the profile thereof, so that the filter guidewire may pass through the treatment area without disturbing any stents or otherwise interfering with the treated vessel.

Semi-permanent filters may also be placed in a blood vessel, and in particular within a vein, for retaining thromboli or blood clots that may form after an interventional procedure and cause an embolism. Generally these semi-permanent filters are in the form of frusto-conical baskets, i.e., centrally-collecting basket filters, that are deployed within a vein downstream from the interventional or surgical procedure, in the blood flow path that is desired to be filtered, which generally is the vena cava proximate the heart to prevent any blood clots or thromboli from reaching the heart.

In conventional distal protection filters, regardless of how the filter is expanded or collapsed during a procedure, i.e., by use of a sheath or by use of a push-pull mechanism, or whether the filter is temporarily or semi-permanently deployed, the filters are designed to capture emboli in the center of the filter, ergo, in the center of the vessel lumen. Blood flow is known to be laminar or spirally laminar such that the highest flow rate is along the center of the blood vessel lumen, with the flow rate diminishing to near zero at the vessel wall. Thus, if a filter collects even a moderate amount of emboli in the center of a blood vessel lumen, then such blockage may impede high-velocity central blood flow, resulting in slow total antegrade flow through the filter. Therefore, a conventional filter having centralized collection need not fill to capacity or completely clog with embolic debris to cause a significant reduction in total blood flow there through. Slow blood flow through an intravascular filter may result in a stagnant debris-containing column of blood in the vessel proximal to the filter. If the debris is not subsequently aspirated, it may present an embolic risk during filter retraction. Further, recent research has linked slow flow during vascular interventions with an increased risk of a post-operative stroke. Thus, what is needed are temporary and semi-permanent intravascular filters that avoid slowing total blood flow by maintaining central blood flow through the filter during a vascular interventional procedure or after semi-permanent deployment within a vessel while providing adequate filter porosity and storage capacity for collected particulate.

### BRIEF SUMMARY OF THE INVENTION

Embodiments of the present invention are directed to temporary or semi-permanent filtration devices for collecting emboli and/or thromboli in a body lumen. The design of the filtration device provides an improved flow rate through a larger central lumen area than is achieved with a standard filter having similar dimensions. Further, a greater volume of embolic material can be captured within a peripheral collection zone of the improved filtration device than can be accumulated in the central collection area of a standard filter.

In an embodiment, a filtering device according to the present invention is positioned proximate a distal end of a distal protection device with a core wire extending there through. The filtering device includes a tubular member having an outer surface for apposition with a vessel wall that defines the body lumen and a filter member positioned within an interior of the tubular member. In various embodiments, the tubular member may be of a mesh material and the filter member may be a braided filter or vice versa, as well each of the tubular member and the filter member may be of a mesh or braided material. The filter member has an outer surface that faces art inner surface of the tubular member, such that an embolic debris collection chamber is defined within the tubular member and outside the filter member. The filtering device may further include proximal and distal self-expanding wave springs positioned within the interior of the tubular member for deploying the filtering device. In an alternate embodiment, the filtering device may be made of a shape memory material and formed to be self-expanding. In operation, a proximal end of the filter member radially disperses the embolic debris toward a periphery of the filtering device for collection in the collection chamber while maintaining continuous blood flow through the center of the body lumen.

In an embodiment, the filtering device has a proximal end coupled to an elongate shaft that is coaxially disposed around the core wire and a distal end coupled to the core wire. Relative movement between the core wire and the elongate shaft transforms the filtering device between the collapsed configuration, such that the wave springs are collapsed, and a deployed configuration, such that the wave springs are expanded to hold the outer surface of the tabular member in apposition with the vessel wall.

In another embodiment, the proximal and distal ends of the filtering device are slidably coupled to the core wire. The filtering device further includes a first actuator wire attached to the proximal end of the filtering device and a second actuator wire attached to the distal end of the filtering device. Distal movement of the first actuator wire together with proximal movement of the second actuator wire slides the proximal and distal ends of the filtering device closer together to transform the filtering device into the deployed configuration.

In another embodiment, the filtering device includes a first and second filament. The first filament slidably encircles and is attached to the proximal end of the filtering device and the second filament slidably encircles and is attached to the distal end of the filtering device. Proximal movement of the first and second filaments radially draws-down the proximal and distal ends of the filtering device, respectively, to collapse the wave springs and transform the filtering device into its collapsed configuration. In another embodiment, a closure filament is spirally wound around the outer surface of the tubular member with a distal end of the closure filament secured to the filtering device. Proximal movement of the closure filament radially draws-down the filtering device to collapse the wave springs and transform the filtering device into its collapsed configuration.

In another embodiment, the distal protection device includes proximal linkages that attach a proximal end of the outer tubular member to each of a slidable proximal pivot hub and a fixed proximal pivot block. The distal protection device also includes distal linkages that attach a distal end of the outer tubular member to each of a slidable distal pivot hub and a fixed distal pivot block. The filtering device is transformed between its collapsed and deployed configuration by movement of the slidable proximal and distal pivot hubs toward their respective fixed distal and proximal pivot blocks to thereby rotate the proximal and distal linkages away from the core wire to allow expansion of the wave springs.

Another embodiment of the present invention is directed to a semi-permanent filtering device for positioning within one of the venae cavae. The filtering device includes a tubular member having an outer surface for apposition with the vena cava and an inner surface defining an interior of the tubular member. A fitter member is disposed within the interior of the tubular member and includes a proximal end defining an apex of the filter member, a distal end secured around its circumference to the inner surface of the tubular member, and a fixed length between the proximal end and the distal end in its deployed configuration. Upon implantation of the filtering device within the vena cava, the apex of the filter member is designed to radially disperses thromboli towards a wall of the tubular member for collection within a peripheral collection chamber defined between the inner surface of the tubular member and an outer surface of the filter member.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is an illustration of a distal protection device in accordance with an embodiment of the present invention deployed within the coronary arterial anatomy.

FIG. 2 is a partial longitudinal sectional view of a filtration device in accordance with an embodiment of the present invention.

FIG. 2A is a transverse cross-sectional view of the filtration device of FIG. 2 along line A-A.

FIG. 3 is a partial longitudinal sectional view of a filtration device in accordance with another embodiment of the present invention.

FIG. 3A is a transverse cross-sectional view of the filtration device of FIG. 3 along line A-A.

FIG. 4 is a partial longitudinal sectional view of a filtration device in accordance with another embodiment of the present invention.

FIG. 5 is a partial longitudinal sectional view of a filtration device in accordance with another embodiment of the present invention.

FIG. 6 is an alternate embodiment of a proximal end of the filtration device of FIG. 4.

FIG. 7 is a partial longitudinal sectional view of a filtration device in accordance with another embodiment of the present invention.

FIG. 7A is a transverse cross-sectional view of the filtration device of FIG. 3 along line A-A.

FIG. 8 is a partial longitudinal sectional view of a filtration device in accordance with another embodiment of the present invention.

FIG. 9 is a side view of the filtration device of FIG. 2 in a collapsed configuration.

FIG. 10 is a partial longitudinal sectional view of a filtration device in accordance with another embodiment of the present invention.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician. In reference to a semi-permanent filter to be positioned in one of the venae cavae, “distal” refers to a position or direction upon implantation near or in a direction toward the heart, whereas “proximal” refers to a position or direction upon implantation distant from or in a direction away from the heart.

The following detailed description is merely exemplary in nature and is not intended to limit the invention or the application and uses of the invention. Although the description of the invention is in the context of treatment of blood vessels such as the coronary, carotid and renal arteries or venae cavae, the invention may also be used in any other body passageways where it is deemed useful. Furthermore, there is no intention to be bound by any expressed or implied theory presented in the preceding technical field, background, brief summary or the following detailed description.

Embodiments of the present invention are directed to temporary distal protection devices for use during minimally invasive procedures, such as vascular interventions or other procedures, where the practitioner desires to capture potentially embolic material that may be dislodged during the procedure. As well, embodiments of the present invention are directed to semi-permanent filtration devices that may be used within one of the venae cavae, such as a filtration device that may be semi-permanently deployed within the superior or inferior vena cava for post-procedure protection against blood clot embolization.

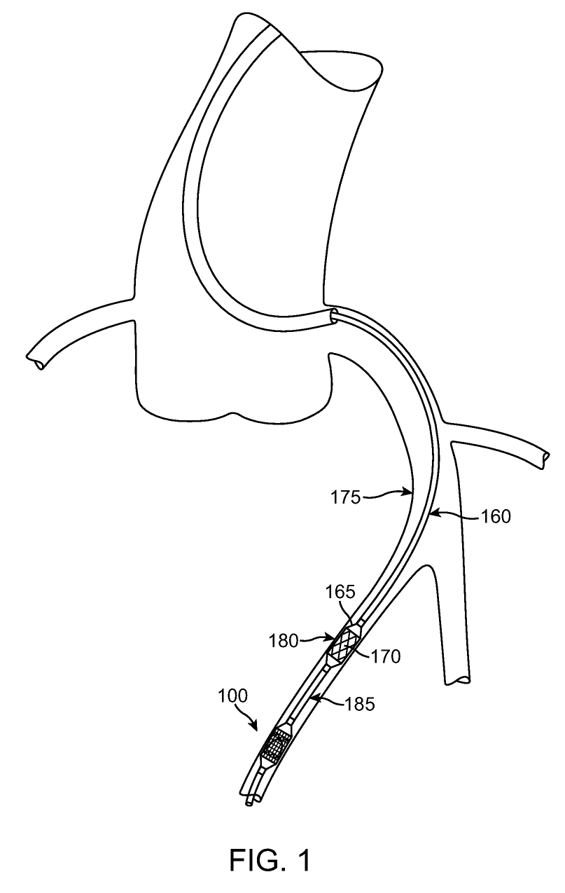

With reference to FIG. 1, deployment of balloon expandable stent 170 is accomplished by threading catheter 160 through the vascular system of the patient until stent 170 is located within a stenosis at predetermined treatment site 180. Once positioned, balloon 165 of catheter 160 is inflated to expand stent 170 against the vascular wail to maintain the opening. Stent deployment can be performed following treatments such as angioplasty, or during initial balloon dilation of the treatment site, which is referred to as primary stenting.

Catheter 160 is typically guided to treatment site 180 by a guidewire. In cases where the target stenosis is located in tortuous vessels that are remote from the vascular access point, such as with the coronary arteries 175, a steerable guidewire is commonly used. According to an embodiment of the present invention, a filter guidewire generally designated as 185 guides catheter 160 to treatment site 180 and includes a distally disposed filtering device 100 according to embodiments of the present invention. Filtering device 100 collects embolic debris that may come loose during the procedure and be carried downstream with blood flowing in an antegrade or proximal-to-distal direction from treatment site 180 through filtering device 100.

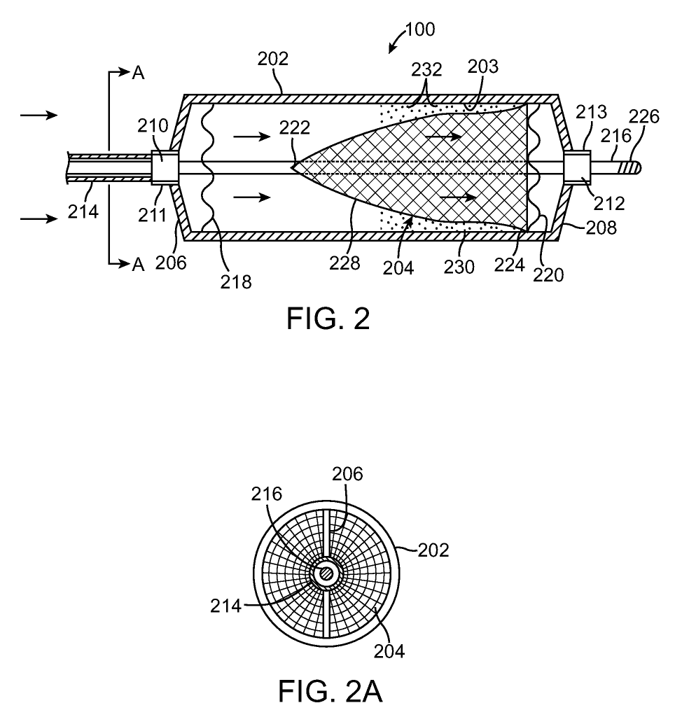

FIG. 2 is a partial longitudinal sectional view of a distal protection device having at its distal end filtering device 100 in accordance with an embodiment of the present invention. Filtering device 100 is shown in a deployed or expanded configuration and includes an outer tubular member 202 having a filter member 204 positioned within its interior to define a collection chamber 230 within outer tubular member 202 and outside of filter member 204. Filtering device 100 is held in its deployed or expanded configuration within the blood vessel by self-expanding proximal and distal wave springs 218, 220 respectively, as discussed further below. Although two wave springs are shown, a greater number of wave springs may be used to insure proper deployment of filtering device 100 without departing from the scope of the present invention. In an alternate embodiment, the entire structure of filtering device 100 may be made self-expanding, e.g., by forming tubular member 202 as a spring-like structure and/or by forming filtering device 100 of a suitable shape memory material, such that wave springs are not needed to deploy the device. The blood flow direction is indicated by the arrows depicted in FIG. 2, such that blood enters filtering device 100 around the proximal hub 210 and exits around the distal hub 212. While the blood flows through an outer surface 228 of filter member 204, embolic debris 232 that impinges on outer surface 228 is redirected by a proximal end or apex 222 of filter member 204 away from the central region of filtering device 100 toward a periphery or inner surface 203 of tubular member 202 for capture. This functionality of filtering device 100 provides a larger, more open central lumen area as compared to a standard, centrally-collecting filter and allows continuous blood flow through a center of the vessel, thereby preventing slow flow conditions from occurring during the interventional procedure. Further, a greater volume of embolic material is captured within the peripheral collection zone of filtering device 100 than can be accumulated in the central collection area of a standard centrally-filling filter made of similar dimensions.

Outer tubular member 202 includes proximal and distal struts 206, 208 attached to respective proximal and distal hubs 210, 212. Proximal and distal struts 206, 208 may be integrally formed with outer tubular member 202, or in an alternate embodiment, may be separate structures that are secured to outer tubular member 202 by laser welding, spot welding, soldering, or a suitable adhesive. In an embodiment, struts 206, 208 may be of nitinol to have a shape memory or high elasticity that aids in the expansion of filtering device 100. In FIG. 2A, two proximal struts 206 are shown connecting outer tubular member 202 to proximal hub 210. Two distal struts 208 are also used to connect outer tubular member 202 to distal hub 212. In further embodiments, more than two proximal and/or distal struts 206, 208 may be utilized.

Outer tubular member 202 may be formed of a mesh material, such as W. L. Gore and Associates' GORE-TEX® expanded polytetrafluoroethylene fabric, or outer tubular member 202 may be formed of expanded polytetrafluoroethylene foam sold by Allergan Inc. under the trademark SOFTFORM™. Alternatively, outer tubular member 202 may be formed from a metal mesh or braided material, such as a mesh or braided material made of nitinol or stainless steel. In an alternate embodiment, outer tubular member 202 and wave springs 218, 220 may be replaced by a stent-graft structure, such as any of those disclosed in U.S. Pat. Nos. 5,824,037, 6,099,559, and 6,193,745, which are incorporated by reference herein in their entirety.

Proximal hub 210 is connected to a distal end of a tubular shaft 214 of filter guidewire 185 and distal hub 212 is connected to core wire 216 of filter guidewire 185. Core wire 216 is coaxlally slidably disposed within tubular shaft 214. Core wire 216 may be made from a super-elastic metals such as nitinol, or a stainless steel wire. In an embodiment of the present invention (not shown), core wire 216 may be tapered at its distal end and/or be comprised of one or more core wire sections. Core wire 216 has a proximal end (not shown) that extends outside of the patient from a proximal end of tubular shaft 214 (not shown), and a coiled distal end 226.

Proximal hub 210 may be either rotatably or fixedly attached to tubular shaft 214, as long as an axial position of proximal hub 210 is fixed with respect to tubular shaft 214. Likewise, distal hub 212 may be either rotatably or fixedly attached to core wire 216, as long as an axial position of distal hub 212 is fixed or has a short, limited range of axial motion with respect to core wire 216. Accordingly in alternate embodiments, proximal arid distal hubs or filter ends 210, 212 may be spot welded, laser welded or secured using a bonding sleeve or adhesive to tubular shaft 214 or core wire 216, respectively, as would be apparent to one skilled in the relevant art. In this embodiment, proximal and distal hubs 210, 212 are surrounded by respective bands 211, 213, which are radiopaque markers to aid in fluoroscopic observation during manipulation of filtering device 100 through a patient's vasculature.

In the embodiment of FIGS. 2 and 2A, filter member 204 is a braided generally conical filter comprised of a plurality of braiding wires or filaments that are woven together. In another embodiment, filter member 204 may be substantially ogival in shape. Braiding wires or filaments for producing braided filter member 204 are preferably made from stainless steel, a pseudo-elastic metal such as nitinol, or a nickel-based super alloy. In another embodiment, filter member 204 may be formed from a suitable mesh or porous material, such as a laser drilled polyurethane membrane having openings of between 50-100 microns. As shown in FIG. 2, filter member 204 may be self-supporting, for e.g., by being made of nitinol to be self-expanding or by being mounted on the medical device so that its length is stretched between its proximal and distal ends. In an alternate embodiment, filter member 204 may include a support structure (not shown), such as a wire frame, that supports a mesh or braided covering. Filter member 204 collects embolic debris on outer surface 228 while permitting fluid to flow there through, such as an amount of blood flow sufficient for perfusion of body tissues. Suitable mesh filters and braided filters are disclosed in U.S. Pat. No. 6,346,116 that is incorporated by reference herein in its entirety.

In alternate embodiments of the present invention, each of tubular member 202 and filter member 204 may be made of either a mesh material or a braided structure, as well as from the same or different materials so as to achieve a desired functionality, such as self-expansion, greater perfusion, better stability, etc., in filtering device 100.

A proximal end or apex 222 of filter member 204 is coupled to core wire 216 and a distal end 224 of filter member 204 is fixedly attached around its circumference to an inner surface 203 of outer tubular member 202 proximate distal wave spring 220. Filter member apex 222 may be fixedly attached to core wire 216, such as by spot welding, laser welding or secured using a bonding sleeve or adhesive, or slidably disposed thereon by way of a sleeve and slop arrangement (not shown). Filter member distal end 224 may be fixedly attached to inner surface 203 of outer tubular member 202 by spot welding, laser welding, suturing, or by using an adhesive. In an alternate embodiment, distal end 224 of filter member 204 may be secured to distal wave spring 220.

Outer surface 228 of filter member 204 has at least a proximal portion thereof treated with a lubricious or slippery coating, e.g., a fluoropolymer or silicone, such that outer surface 228 prevents debris from lodging across relatively centrally-located pores of filter membrane 204. Due to the generally acute angle formed between the outer surface 228 of liter member 204 and the axial path of blood flowing there through, the proximal portion of filter member 204 acts as a slide for moving particulate dispersed or redirected by apex 222 radially outward and distally for accumulation within an annular debris collection zone or pocket 230, which is formed between the distal inner surface 203 of outer tubular member 202 and the distal outer surface 228 of filter member 204.

The initial capture of particulate has minimal effect on total blood flow through filtering device 100 because the trapped material accumulates in collection zone 230 adjacent outer tubular member 202 and near the blood vessel wall where laminar blood flow is already slow. The centrally-located pores of filter member 204 will begin to become obstructed only after collection zone 230 has been filled with collected debris and if particulate continues to accumulate in the remainder of the collection chamber proximal to collection zone 230. Filtering device 100 thus maintains an effective total blood flow as long as possible because the slowest-flowing portion of blood is obstructed first and the fastest-flowing portion of blood is obstructed last, if at all. Additionally, the filtering device 100 offers greater volumetric efficiency by capturing more emboli or thromboli than a similarly dimensioned standard centrally-collecting basket filter prior to requiring aspiration.

Filtering device 100 is expanded distal of the vascular treatment site by operating the push-pull mechanism as follows. Axial movement of core wire 216 with respect to hollow shaft 214 moves proximal and distal hubs or filter ends 210, 212 closer together, thereby permitting wave springs 218, 220 to self-expand. Expanded wave springs 218, 220 push outer tubular member 202 into apposition with the vessel wall (see FIG. 1) and expand filter member 204 into its generally conical deployed configuration. In its deployed configuration, a length between proximal end 222 and distal end 224 of filter member 204 is fixed, such that the apex or proximal end 222 of filter member 204 has a substantially fixed axial position relative to outer member 202. Filtering device 100 is thereafter held in its deployed or expanded configuration by proximal and distal wave springs 218, 220, such that circumferential surface contact is maintained between outer tubular member 202 and the vessel wall to prevent any emboli from slipping past filtering device 100 during the procedure. In embodiments of the present invention, wave springs 218, 220 may each be similar to a single module of a self-expanding stent such as any of those disclosed in U.S. Pat. No. 5,817,152 that is incorporated by reference herein in its entirety, or a single module of a self-expanding stent graft such as any of those disclosed in U.S. Pat. Nos. 5,824,037, 6,099,559, and 6,193,745, which are incorporated herein above. Wave springs 218, 220 may be made of nitinol, stainless steel, a cobalt chromium alloy or other material that provides the requisite elasticity needed in wave springs 218, 220 to perform in accordance with embodiments of the present invention.

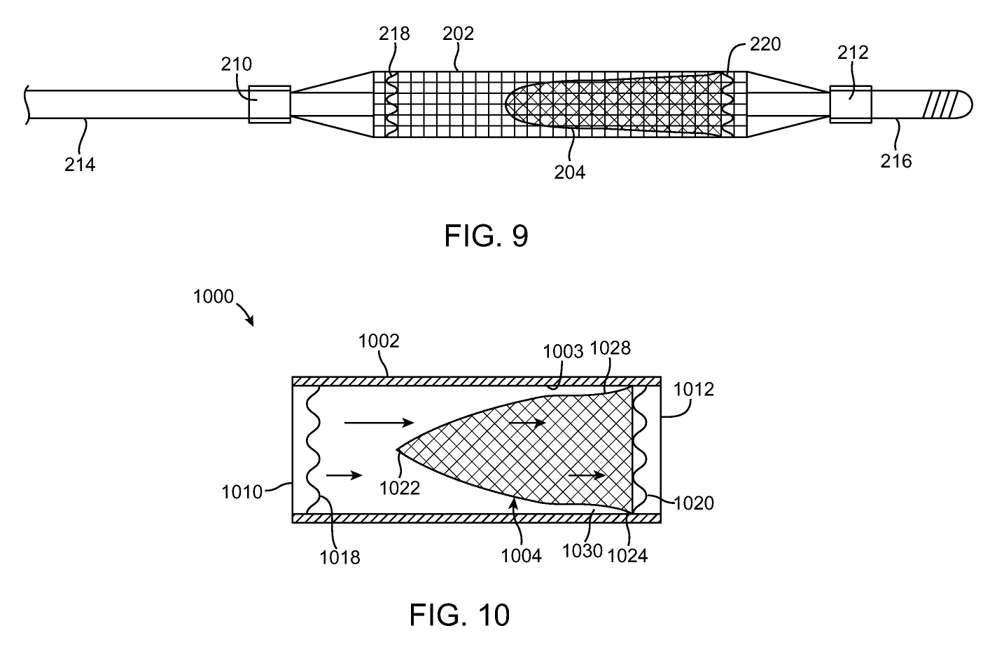

When an intravascular treatment is complete, filtering device 100, which may now contain embolic debris if aspiration has not been performed, must be collapsed and removed from the patient. Filtering device 100 is mechanically collapsed by the push-pull mechanism previously discussed above into a collapsed configuration as shown in FIG. 9, wherein wave springs 218, 220 of filtering device 100 are compressed by the draw-down of outer tubular member 202 due to the distal movement of core wire 216 relative to tubular shaft 214. The filtering device 100 is then retracted from the patient's vasculature.

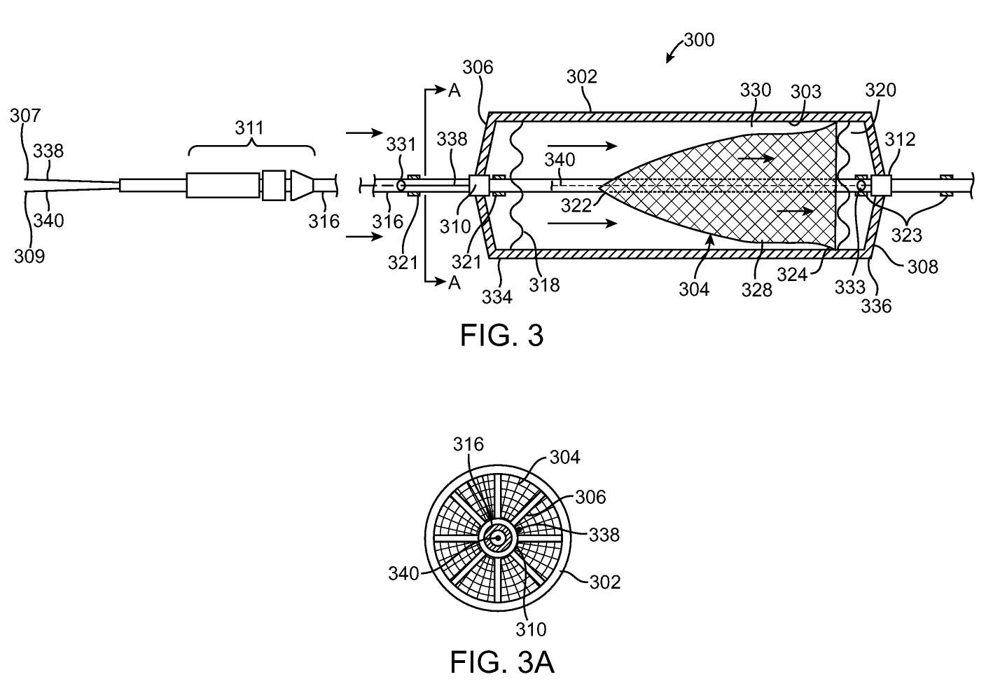

FIGS. 3 and 3A illustrate another embodiment of the present invention. Filtering device 300 includes an outer cylindrical member 302 with an inner filter member 304 having a proximal apex 322 for radially redirecting embolic debris within the blood flow (see arrows) toward a collection zone 330 situated between a distal portion of inner surface 303 of outer cylindrical member 302 and a distal portion of outer surface 328 of inner filter member 304. Accordingly, the principle of operation of filtering device 300 is similar to that of filtering device 100.

Filtering device 300 differs from push-pull operated filtering device 100 in the manner in which it is expanded and collapsed. In the embodiment of FIGS. 3 and 3A, both proximal end 310 and distal end 312 of filtering device 300 are slidably coupled to core wire 316. Each of proximal and distal ends 310, 312 is also attached by a plurality of proximal and distal links 306, 308 to a respective proximal and distal end 334, 336 of outer cylindrical member 302. Proximal and distal links 306, 308 may be made from materials similar to those of struts 206, 208 described above, or may be made integral with outer member 302. In an alternate embodiment, links 306, 308 may be threads or cords. Although eight links are shown, fewer or more links may be used without departing from the scope of the present invention.

Proximal end 310 of filtering device 300 is attached to a first actuator wire or rod 338 and distal end 312 of filtering device 300 is attached to a second actuator wire or rod 340. First and second actuator wires 338, 340 extend proximally from proximal and distal filter ends 310, 312, respectively, for a length external of core wire 316 and then enter an interior of core wire 316 at apertures 331, 333, respectively, such that first and second actuator wires 338, 340 slidably extend therein. Proximal ends 307, 309 of first and second actuator wires 338, 340 extend out of the patient proximal of a hub 311 and may be secured to and actuated by an actuation mechanism (not shown), as would be apparent to one of ordinary skill in the art.

To expand filtering device 300, first actuator wire 338 is moved distally to slide proximal end 310 distally and thereby radially extend or provide slack in proximal links 306 to permit the release and expansion of self-expanding proximal wave spring 318. Simultaneously, second actuator wire 340 is moved proximally to slide distal end 312 proximally and thereby radially extend or provide slack in distal links 308 to permit the release and expansion of self-expanding distal wave spring 320. To collapse filtering device 300, the movement of each of first and second actuator wires 338, 340 is reversed such that proximal and distal ends 310, 312 of filtering device 300 are moved apart collapsing outer cylindrical member 302 about proximal and distal wave springs 318, 320. Optionally as shown in FIG. 3, a set of proximal and distal stops 321, 323 are positioned proximal and distal of each end 310, 312 to prevent over expansion of, or inadvertent damage to filtering device 300. As in the previous embodiment, proximal end or apex 322 of internal filter member 304 may be fixedly attached to core wire 316, such as by spot welding, laser welding or secured using a bonding sleeve or adhesive, or slidably disposed thereto by way of a sleeve and stop arrangement (not shown). Further, distal end 324 of internal filter member 304 is attached about its periphery to inner surface 303 of outer cylindrical member 302 in a manner as described above with reference to the previous embodiment. As in the previous embodiment, a working length between proximal end 322 and distal end 324 of filter member 304 is fixed in its deployed configuration.

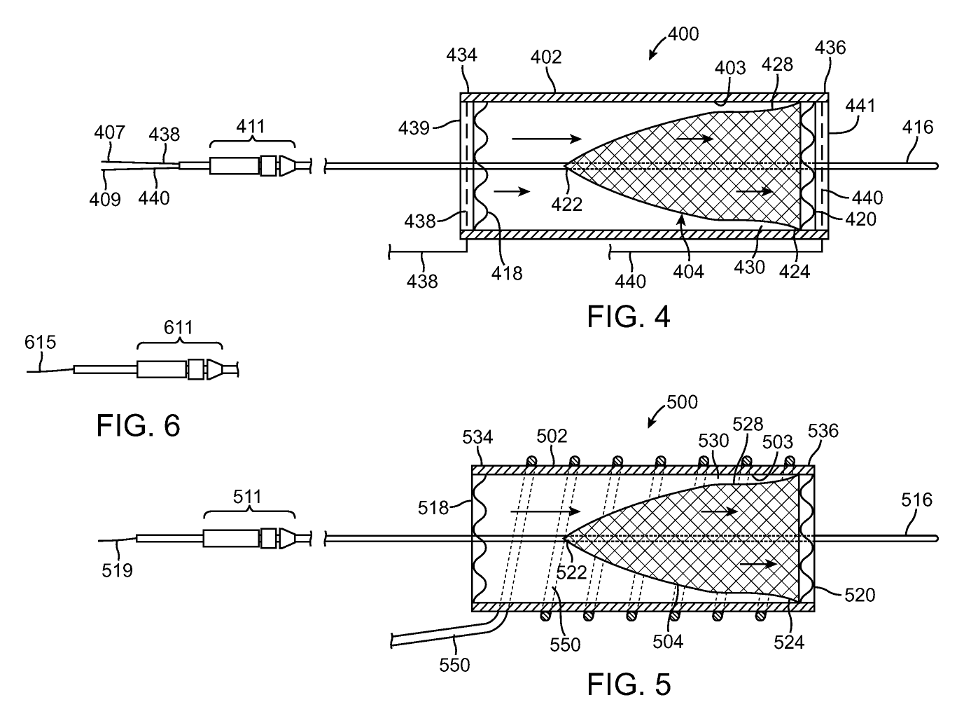

FIG. 4 is a partial longitudinal sectional view of a distal protection device 400 in accordance with another embodiment of the present invention. Filtering device 400 includes an outer mesh member 402 with an inner filter member 404 having a proximal end 422 for radially redirecting embolic debris within the blood flow (see arrows) toward a periphery of the filtering device. As such, embolic debris accumulates in a collection area 430 situated between a distal portion of inner surface 403 of outer mesh member 402 and a distal portion of outer surface 428 of inner filter member 404. Accordingly, the principle of operation of filtering device 400 is similar to that of filtering device 100.

Filtering device 400 differs from filtering device 100 in the manner in which it is expanded and collapsed. In the embodiment of FIG. 4, proximal end 434 of outer mesh member 402 includes a folded or hem-like portion 439 that slidably contains a first filament 438, which has a distal end fixedly secured to filtering device 400 and a proximal end 407 that extends outside the patient proximal of a hub 411. Similarly, distal end 436 of outer mesh member 402 includes a folded or hem-like portion 441 that slidably contains a second filament 440, which has a distal end fixedly secured to filtering device 400 and a proximal end 409 that extends outside the patient proximal of hub 411. In another embodiment, first and second filaments 438, 440 may be loosely woven through the circumference of proximal and distal ends 434, 436 of outer mesh member 402, respectively, in such a manner that each is readily slidable there through. The first and second filaments 438, 440 may be pulled proximally to radially draw-down or close respective proximal and distal ends 434, 436 of outer mesh member 402 in a “purse string” like manner. When first and second filaments 438, 440 are pulled proximally, proximal and distal wave springs 418, 420 are collapsed by the radial draw-down of proximal and distal ends 434, 436 of outer mesh member 402. Similarly to expand or deploy filtering device 400, first and second filaments 438, 440 are allowed slack or released so that radial expansion of self-expanding wave springs 418, 420 may pull distally a sufficient length of first and second filaments 438, 440 to thereby permit deployment of the wave springs 418, 420, which subsequently push proximal and distal ends 434, 436 of outer mesh member 402 into apposition with the vessel wall. Although a filament is shown at both the proximal and distal ends of filtering device 400, the device may include a closure filament at the proximal end only, such that filtering device 400 may be collapsed or expanded by a single filament. In an alternate embodiment as shown in FIG. 6, first and second filaments 438, 440 may be joined proximal of filtering device 400 into a single filament 615 that exits proximal of a hub 611, such that filament 615 may be manipulated to push-pull first and second filaments 438, 440 and permit expansion and collapse of wave springs 418, 420 as previously described.

In the embodiment of FIG. 4, proximal end or apex 422 of internal filter member 404 is fixedly attached to core wire 416, such as by spot welding, laser welding or secured using a bonding sleeve or adhesive. Further, distal end 424 of internal filter member 404 is attached about its periphery to inner surface 403 of outer mesh member 402 in a manner as described above with reference to the embodiment of FIG. 2. As in the previous embodiments, a working length between proximal end 422 and distal end 424 of filter member 404 is fixed in its deployed configuration.

FIG. 5 is a partial longitudinal sectional view of a distal protection device 500 in accordance with another embodiment of the present invention. Filtering device 500 includes an outer mesh member 502 with an inner filter member 504 having a proximal end 522 for radially redirecting embolic debris within the blood flow (see arrows) toward a periphery of the filtering device for accumulation within a plaque collection zone 530 situated between a distal portion of inner surface 503 of outer mesh member 502 and a distal portion of outer surface 528 of inner filter member 504. Accordingly, the principle of operation of filtering device 500 is similar to that of filtering device 100.

Filtering device 500 differs from filtering device 100 in the manner in which it is expanded and collapsed. In the embodiment of FIG. 5, outer mesh member 502 includes a closure filament 550 with a length spirally wrapped around outer mesh member 502. A distal end of closure filament 550 is fixed proximate distal end 536 of outer mesh member 502 with a proximal end 519 extending outside a patient proximal of a hub 511. The spirally wrapped length of closure filament 550 may be held in a channel or groove (not shown) in an outer surface of outer mesh member 502. In another embodiment, the spirally wound length of closure filament 550 may be loosely woven through outer mesh member 502 in such a manner that closure filament 550 is readily slidable there through. To collapse filtering device 500, the proximal end 519 of closure filament 550 may be pulled proximally to radially draw-down or close outer mesh member 502. When closure filament 550 is pulled proximally, proximal and distal wave springs 518, 520 are collapsed by the radial draw-down of outer mesh member 502. Similarly to expand or deploy filtering device 500, closure filament 550 is allowed slack or released so that radial expansion of self-expanding wave springs 518, 520 will pull distally a sufficient length of closure filaments 550 to thereby permit deployment of wave springs 518, 520, which subsequently push proximal and distal ends 534, 536 of outer mesh member 502 into apposition with the vessel wall.

In the embodiment of FIG. 5, proximal end or apex 522 of internal filter member 504 is fixedly attached to core wire 516, such as by spot, welding, laser welding or secured using a bonding sleeve or adhesive. Further, distal end 524 of internal filter member 504 is attached about its periphery to inner surface 503 of outer mesh member 502 in a manner as described above with reference to the embodiment of FIG. 2. As in the previous embodiments, a working length between proximal end 522 and distal end 524 of filter member 504 is fixed in its deployed configuration.

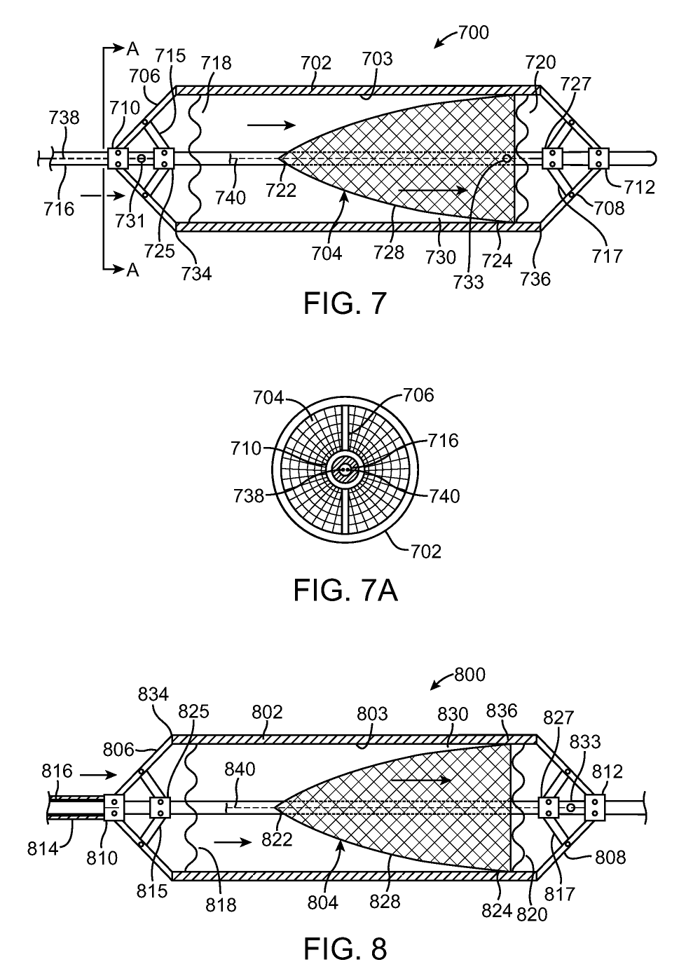

FIGS. 7 and 7A illustrate another embodiment of the present invention. Filtering device 700 includes an outer cylindrical member 702 with an inner filter member 704 having a proximal apex 722 for radially redirecting embolic debris within the blood flow (see arrows) toward a collection zone 730 situated between a distal portion of inner surface 703 of outer cylindrical member 702 and a distal portion of outer surface 728 of inner filter member 704. Accordingly, the principle of operation of filtering device 700 is similar to that of filtering device 100.

Filtering device 700 differs from push-pull operated filtering device 100 in the manner in which it is expanded and collapsed. In the embodiment of FIGS. 7 and 7A, both proximal end 710 and distal end 712 of filtering device 700 are pivot blocks fixed to core wire 716 and provide pivot points for a pivotable connection with proximal and distal primary linkages 706, 708. Proximal and distal primary linkages 706, 708 in turn connect to respective proximal and distal ends 734, 736 of outer cylindrical member 702. Proximal, and distal primary linkages 706, 708 also include pivot points for pivotable attachment of secondary linkages 715, 717, respectively, which in turn are pivotally attached at pivot points to proximal and distal pivot hubs 725, 727. Proximal and distal pivot hubs 725, 727 are slidably coupled to core wire 716. As well, proximal pivot hub 725 is attached to a first actuator wire or rod 738 and distal pivot hub 727 is attached to a second actuator wire or rod 740. First and second actuator wires 738, 740 extend proximally from pivot hubs 725, 727 for a length external of core wire 716 and then enter an interior of core wire 716 at apertures 731, 733, respectively, such that first and second actuator wires 738, 740 slidably extend therein. Proximal ends of first and second actuator wires 738, 740 extend out of the patient, in a manner as shown in FIG. 3, and may be secured to and actuated by an actuation mechanism (not shown), as would be apparent to one of ordinary skill in the art.

To expand filtering device 700, first actuator wire 738 is pulled proximally to slide proximal pivot hub 725 proximally toward fixed proximal end 710 of filtering device 700. Proximal movement of proximal pivot hub 725 thereby rotates proximal secondary linkages 715 away from core wire 716 to radially extend proximal primary linkages 706 to permit the release and expansion of self-expanding proximal wave spring 718. Simultaneously, second actuator wire 740 is moved distally to slide distal pivot hub 727 distally toward fixed distal end 712 of filtering device 700. Distal movement of distal pivot hub 727 thereby rotates distal secondary linkages 717 away from core wire 716 to radially extend primary linkages 708 to permit the release and expansion of self-expanding distal wave spring 720. To collapse filtering device 700, the movement of each of first and second actuator wires 738, 740 is reversed such that proximal and distal pivot hubs 725, 727 are moved toward each other rotating secondary linkages 715, 717 toward core wire 716, which in turn pulls primary linkages 706, 708 toward core wire 716, to collapse outer cylindrical member 702 about proximal and distal wave springs 718, 720. As in the previous embodiment, proximal end or apex 722 of internal filter member 704 may be fixedly attached to core wire 716, such as by spot welding, laser welding or secured using a bonding sleeve or adhesive, or slidably disposed thereto by way of a sleeve and stop arrangement (not shown). Further, distal end 724 of internal filter member 704 is attached about its periphery to inner surface 703 of outer cylindrical member 702 in a manner as described above with reference to the previous embodiment. As in the previous embodiments, a working length between proximal end 722 and distal end 724 of filter member 704 is fixed in its deployed configuration.

FIG. 8 illustrates another embodiment of the present invention. Filtering device 800 Includes a cylindrical member 802 with an inner filter member 804 having a proximal apex 822 for radially redirecting embolic debris within the blood flow (see arrows) toward a collection zone 830 situated between a distal portion of inner surface 803 of cylindrical member 802 and a distal portion of outer surface 828 of filter member 804. Accordingly, the principle of operation of filtering device 800 is similar to that of filtering device 100.

Filtering device 800 differs from push-pull operated filtering device 100 in the manner in which it is expanded and collapsed. In the embodiment of FIG. 8, proximal pivot hub or end 810 of filtering device 800 is connected to a distal end of tubular shaft 814 and distal pivot hub or end 812 of filtering device 800 is slidably coupled to core wire 816. Core wire 816 is coaxially slidably disposed within tubular shaft 814. As well, distal pivot hub 812 is attached to an actuator wire or rod 840. Actuator wire 840 extends for a length external of core wire 816 and then enters an interior of core wire 816 at aperture 833, such that actuator wire 840 slidably extends therein. A proximal end of actuator wire 840 extends out of the patient, in a manner as shown in FIG. 3, and may be secured to and actuated by an actuation mechanism (not shown), as would be apparent to one of ordinary skill in the art.

Both proximal pivot hub 810 and distal pivot hub 812 include pivot points for pivotally connecting proximal and distal primary linkages 806, 808. Proximal and distal primary linkages 806, 808 in turn connect to respective proximal and distal ends 834, 836 of cylindrical member 802. Proximal and distal primary linkages 806, 808 also include pivot points for pivotal attachment of secondary linkages 815, 817, respectively, which in turn are attached to proximal and distal pivot blocks 825, 827 at pivot connections thereon. Proximal and distal pivot blocks 825, 827 are fixedly attached to core wire 816.

To expand filtering device 800, tubular shaft 814 is pushed distally to slide proximal hub 810 distally toward fixed proximal pivot block 825. Distal movement of proximal hub 810 thereby rotates proximal primary linkage 806 away from core wire 816 to radially extend proximal secondary linkages 815 to permit the release and expansion of self-expanding proximal wave spring 818. Simultaneously, actuator wire 840 is pulled proximally to slide distal hub 812 proximally toward fixed distal pivot block 827. Proximal movement of distal hub 812 thereby rotates distal primary linkages 808 away from core wire 816 to radially extend secondary linkages 817 to permit the release and expansion of self-expanding distal wave spring 820. To collapse filtering device 800, the movement of each of tubular shaft 814 and actuator wire 840 is reversed such that proximal and distal hubs 825, 827 are moved away from each other rotating primary linkages 806, 808 toward core wire 816, which in turn pulls secondary linkages 815, 817 toward core wire 816, to collapse outer cylindrical member 802 about proximal and distal wave springs 818, 820. As in the previous embodiment, proximal end or apex 822 of internal filter member 804 may be fixedly attached to core wire 816, such as by spot welding, laser welding or secured using a bonding sleeve or adhesive, or slidably disposed thereto by way of a sleeve and stop arrangement (not shown). Further, distal end 824 of internal filter member 804 is attached about its periphery to inner surface 803 of outer cylindrical member 802 in a manner as described above with reference to the previous embodiment. As in the previous embodiments, a working length between proximal end 822 and distal end 824 of filter member 804 is fixed in its deployed configuration.

FIG. 10 is a partial longitudinal sectional view of a semi-permanent filtering device 1000 in accordance with another embodiment of the present invention. Filtering device 1000 is shown in a deployed or expanded configuration and includes an outer tubular member 1002 having a braided filter member 1004 positioned within its interior to define a collection zone 1030 within outer tubular member 1002 and outside of filter member 1004. Filtering device 1000 is held in its deployed or expanded configuration within the blood vessel by self-expanding proximal and distal wave springs 1018, 1020 respectively, as previously discussed. Although two wave springs are shown, a greater number of wave springs may be used to insure proper deployment of filtering device 1000 without departing from the scope of the present invention. In an alternate embodiment, the entire structure of filtering device 1000 may be made self-expanding, e.g., by forming tubular member 1002 as a spring-like structure and/or by forming filtering device 1000 of a suitable shape memory material, such that wave springs are not needed to deploy the device.

The blood flow direction is indicated by the arrows depicted in FIG. 10, such that when filtering device 1000 is send-permanently positioned within one of the venae cavae the arrows indicate that the blood flow is towards the heart. Accordingly, blood flow enters filtering device 1000 at proximal end 1010 and exits through distal end 1012, wherein distal end 1012 is positioned closer to the heart than proximal end 1010. While the blood flows through an outer surface 1028 of filter member 1004, thromboli that impinges on outer surface 1028 is redirected by a proximal end or apex 1022 of filter member 1004 away from the central region of filtering device 1000 toward a periphery or inner surface 1003 of tubular member 1002 for capture. This functionality of filtering device 1000 provides a larger, more open central lumen area as compared to a standard, centrally-collecting filter and allows continuous blood flow through a center of the vessel, thereby providing an increased flow rate over the standard filter. Further, a greater volume of thromboli may be captured within the peripheral collection zone of improved filtration device 1000 than can tie accumulated in the central collection area of a standard filter made of similar dimensions.

Filter member distal end 102.4 may be fixedly attached to inner surface 1003 of outer tubular member 1002 by spot welding, laser welding, suturing or by using an adhesive. Outer surface 1028 of filter member 1004 may have at least a proximal portion thereof treated with a lubricious or slippery coating, e.g., a fluoropolymer or silicone, such that outer surface 1028 prevents debris from lodging across relatively centrally-located pores of filter membrane 1004. Due to the generally acute angle formed between the outer surface 1028 of filter member 1004 and the axial path of blood flowing there through, the proximal portion of filter member 1004 acts as a slide for moving particulate dispersed or redirected by apex 1022 radially outward and distally for accumulation within the annular debris collection zone or pocket 1030, which is formed between the distal, inner surface 1003 of outer tubular member 1002 and the distal outer surface 1028 of filter member 1004.

Filtering device 1000 is delivered to one of the venae cavae by an appropriate delivery catheter, such as a sheath catheter that will maintain filtering device 1000 in its compressed configuration until it is deployed within the body lumen. In embodiments that utilize wave springs 1018, 1020, the wave springs will push outer tubular member 1002 into apposition with the vessel wail to expand filter member 1004 into its generally conical deployed configuration shown in FIG. 10. In its deployed configuration, a length between proximal end 1022 and distal end 1024 of filter member 1004 is fixed, such that apex 1022 of filter member 1004 has a substantially fixed axial position relative to outer member 1002. Filtering device 1000 is thereafter held in its deployed or expanded configuration by proximal and distal wave springs 1018, 1020, such that circumferential surface contact is maintained between outer tubular member 1002 and the vessel wall to prevent migration of filtering device 1000.

Although two wave springs are shown in each of the foregoing embodiments, a greater number of wave springs may be used to insure proper deployment of any of the filtering device without departing from the scope of the present invention. Alternatively, a portion or entire structure of any of the previously described filtering devices may be made self-expanding, such that wave springs are not needed to deploy the device.

A filtering device in accordance with the present invention may be transformable between its collapsed and expanded configurations by relative movement between its ends. Such movement may be accomplished by a filter guidewire mechanism similar to that shown in any of the filter guidewires disclosed in U.S. Pat. No. 6,706,055, U.S. Pat. No. 6,818,006 and U.S. Pat. No. 6,866,677, which are incorporated by reference herein in their entireties. Alternatively, a filtering device in accordance with the present invention may be deployed and/or retrieved via a sheath catheter, such as by the method and apparatus disclosed in U.S. Pat. No. 6,059,814, which is incorporated by reference herein in its entirety, or the '116 patent previously incorporated by reference herein. The transformation of the filtering device may be impelled by external mechanical means alone or by self-shaping memory (either self-expanding or self-collapsing) within the filtering device. In embodiments of the present invention, the filter devices are self-expanding which means they have a mechanical memory to return to the expanded or deployed configuration. Such mechanical memory can be imparted to the metal comprising filter members 204, 304, 404, 504, 704, 804, 1004 and/or tubular members 202, 302, 402, 502, 702, 802, 1002 by thermal treatment to achieve a spring temper in stainless steel, for example, or to set a shape memory in a susceptible metal alloy, such as nitinol. In such embodiments, it is preferable that at least the majority of braiding wires or filaments forming the braided filter and/or tubular members be capable of being heat treated into the desired filter shape/component, and such wires should also have sufficient elastic properties to provide the desired self-expanding or self-collapsing features.

Optionally, radiopaque markers, as shown in FIG. 2, may be placed on the proximal and distal ends of filtering devices 300, 400, 500, 700, 800, 1000 to aid in fluoroscopic observation during manipulation thereof. Alternatively, fluoroscopic visualization of the filtering devices may be enhanced when at least one of the filaments includes a wire having enhanced radiopacity compared to conventional non-radiopaque wires suitable for braiding filter members 204, 304, 404, 504, 704, 804, 1004 and/or tubular members 202, 302, 402, 502, 702, 802, 1002. Braiding wires or filaments having enhanced radiopacity may be made of, or coated with a radiopaque metal such as gold, platinum, tungsten, alloys thereof, or other biocompatible metals having a relatively high X-ray attenuation coefficient compared with stainless steel or nitinol One or more filaments having enhanced radiopacity may be inter-woven with non-radiopaque wires, or all wires comprising filter members 204, 304, 404, 504, 704, 804, 1004 and/or tubular members 202, 302, 402, 502, 702, 802, 1002 may have the same enhanced radiopacity.

Alternatively, one or more of braiding wires/braid filaments may comprise a composite wire having a radiopaque core and non-radiopaque layer or casing. Such coaxial, composite wires are referred to as DFT (drawn-filled-tube) wires in the metallic arts, and filters comprising such wires are disclosed in U.S. Pat. No. 6,866,677, reference herein above.

While various embodiments according to the present invention have been described above, it should be understood that they have been presented by way of illustration and example only, arid not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A filtering apparatus for collecting embolic debris in a body lumen, comprising:
a core wire extending from a proximal end to a distal end of the distal protection device; and
a filtering device positioned proximate the distal end of the distal protection device with the core wire extending there through, the filtering device including
a tubular member having an outer surface for apposition with a vessel wall defining the body lumen and an inner surface defining an interior of the tubular member, and
a filter member positioned within the interior of the tubular member, the filter member having a proximal end coupled to the core wire, a distal end attached to the tubular member, and an outer surface facing the inner surface of the tubular member, wherein a collection chamber is defined within the tubular member and outside the filter member.

2. The filtering apparatus of claim 1, wherein the proximal end of the filter member radially disperses the embolic debris to permit continuous blood flow through the center of the body lumen.

3. The filtering apparatus of claim 1, wherein the filter member is a braided filter.

4. The filtering apparatus of claim 3, wherein the braided filter is ogival.

5. The filtering apparatus of claim 3, wherein the braided filter is conical.

6. The filtering apparatus of claim 3, wherein the tubular member is of a mesh material.

7. The filtering apparatus of claim 6, wherein the mesh material is expanded polytetrafluoroethylene.

8. The filtering apparatus of claim 1, wherein the filtering device further includes proximal and distal self-expanding wave springs within the interior of the tubular member for deploying the filtering device.

9. The filtering apparatus of claim 8, wherein relative longitudinal movement between a proximal end and a distal end of the filtering device accompanies transformation of the filtering device between a collapsed configuration, such that the wave springs are collapsed, and a deployed configuration, such that the wave springs are expanded to hold the outer surface of the tubular member in apposition with the vessel wall.

10. The filtering apparatus of claim 9, further comprising:
an elongate shaft coaxially disposed around the core wire with a proximal end extending out of the body lumen and a distal end coupled to the proximal end of the filtering device, wherein the distal end of the filtering device is coupled to the core wire such that relative movement between the core wire and the elongate shaft transforms the filtering device between the collapsed and deployed configurations.

11. The filtering apparatus of claim 9, wherein the proximal and distal ends of the filtering device are slidably coupled to the core wire.

12. The filtering apparatus of claim 11, further comprising:
a first actuator wire attached to the proximal end of the filtering device; and
a second actuator wire attached to the distal end of the filtering device, wherein distal movement of the first actuator wire together with proximal movement of the second actuator wire slides the proximal and distal ends of the filtering device closer together to transform the filtering device into the deployed configuration.

13. The filtering apparatus of claim 8, further comprising:
a first filament slidably encircling a proximal end of the filtering device, the first filament having a distal end secured to the filtering device and a proximal end extending out of the body lumen; and
a second filament slidably encircling a distal end of the filtering device, the second filament having a distal end secured to the filtering device and a proximal end extending out of the body lumen, wherein proximal movement of the first and second filaments radially draws-down the proximal and distal ends of the filtering device, respectively, to collapse the wave springs and transform the filtering device into its collapsed configuration.

14. The filtering apparatus of claim 13, wherein the proximal ends of the first and second filaments are joined into a single filament that extends out of the body lumen.

15. The filtering apparatus of claim 8, further comprising:
a closure filament spirally wound around the outer surface of the tubular member, the closure filament having a distal end secured to the filtering device and a proximal end that extends out of the body lumen, wherein proximal movement of the closure filament radially draws-down the filtering device to collapse the wave springs and transform the filtering device into its collapsed configuration.

16. The filtering apparatus of claim 15, wherein the closure filament is slidably received within a spiral channel or groove in the outer surface of the tubular member.

17. The filtering apparatus device of claim 1, wherein at least a portion of the filter member is coated with a lubricious coating.

18. The filtering apparatus of claim 8, further comprising:
proximal linkages for attaching a proximal end of the outer tubular member to each of a slidable proximal pivot hub and a fixed proximal pivot block; and
distal linkages for attaching a distal end of the outer tubular member to each of a slidable distal pivot hub and a fixed distal pivot block, wherein movement of the slidable proximal and distal pivot hubs toward the respective fixed distal and proximal pivot blocks rotates the proximal and distal linkages away from the core wire to transform the filtering device into the deployed configuration.

19. The filtering apparatus of claim 18, further comprising:
a first actuator wire attached to the slidable proximal pivot hub; and
a second actuator wire attached to the slidable distal pivot hub, wherein proximal movement of the first actuator wire together with distal movement of the second actuator wire slides the proximal and distal pivot hubs toward the respective fixed proximal and distal pivot blocks to transform the filtering device into the deployed configuration.

20. The filtering apparatus of claim 18, further comprising:
a tubular shaft attached to the slidable proximal pivot hub; and
an actuator wire attached to the slidable distal pivot hub, wherein distal movement of the tubular shaft relative to the core wire together with proximal movement of the actuator wire slides the proximal and distal pivot hubs toward the respective fixed proximal and distal pivot blocks to transform the filtering device into the deployed configuration.

21. The filtering apparatus of claim 3, wherein in a deployed configuration a length between the proximal end and the distal end of the filter member is fixed.

22. The filtering apparatus of claim 3, wherein in a deployed configuration the proximal end of the filter member has a substantially fixed axial position relative to the tubular member.

23-42. (canceled)
